(19) United States
(12) Patent Application Publication
(10) Pub. No.: US 2017/0164815 A1
(43) Pub. Date: Jun. 15, 2017

(54) INFLATABLE BALLOON HOOD

(71) Applicant: Boston Scientific Scimed, Inc., Maple Grove, MN (US)

(72) Inventors: Paul Smith, Smithfield, RI (US); Kevin James McElwee, Franklin, MA (US); Ray Hewenson Tong, Milford, MA (US)

(21) Appl. No.: 15/375,691

(22) Filed: Dec. 12, 2016

Related U.S. Application Data
(60) Provisional application No. 62/265,520, filed on Dec. 10, 2015.

(51) Int. Cl. A61B 1/00; A61B 1/273; A61M 25/10

(52) U.S. Cl. CPC A61B 1/00082; A61B 1/00089; A61B 1/00098; A61M 25/1018; A61B 1/00128; A61B 1/273

(57) ABSTRACT

The present disclosure relates to the field of endoscopy. In particular, the present disclosure relates to systems and methods for navigating the tortious anatomy of the gastrointestinal (GI) tract and visualizing lesions and/or tumors obscured within folds of the lumen wall.

### CROSS-REFERENCE TO RELATED APPLICATIONS

This application claims the benefit of priority to U.S. Provisional Application Ser. No. 62/265,520, filed Dec. 10, 2015, the disclosure of which is herein incorporated by reference in its entirety.

### FIELD

The present disclosure generally relates to the field of endoscopy and specifically, to systems and methods for visualizing lesions and/or tumors within the gastrointestinal (GI) tract. In particular, the present disclosure relates to an extendable and retractable endoscopic hood that provides enhanced visibility as compared to an endoscope alone without interfering with the ability to navigate the endoscope through the narrow strictures and tortuous anatomy of the GI tract.

### BACKGROUND

Endoscopes are commonly used to detect colorectal cancers within the gastrointestinal (GI) tract during a colonoscopy procedure. Some lesions and/or tumors are relatively easy to visualize due to their raised profile and/or large size. However, many lesions and/or tumors include a flat or depressed profile that makes them difficult to identify. This is especially true when these lesions and/or tumors are small and/or hidden within folds of the lumen wall. Visibility may be improved by incorporating an opaque or transparent hood on the distal end of the endoscope, which allows the folds of the lumen wall to be exposed. Unfortunately, endoscopic hoods dramatically reduce the ability of the endoscope to navigate the tortuous anatomy of the GI tract. For example, retroflexion in the colon may be more difficult with an endoscopic hood. In some instances, the endoscopic hoods may also collect feces/debris that obstructs visibility. Once visibility is obstructed, the endoscope must be withdrawn from the patient to clear the feces/debris, thereby prolonging the procedure and increasing the risk of harm to the patient. If strict bowel preparation procedures are not followed, many colonoscopy procedures must be prematurely terminated or canceled altogether.

### SUMMARY

The present disclosure provides an extendable/retractable endoscopic hood that allows physicians to more readily identify small, flat and/or depressed lesions and/or tumors.

In one embodiment, the present disclosure provides an endoscopic system comprising an endoscope comprising an elongate body having a proximal end and a distal end; an endoscopic hood coupled to the distal end of the endoscope, the endoscopic hood comprising a proximal end, a distal end, and a lumen extending therebetween, wherein the lumen is configured to receive the distal end of the endoscope; a flexible inflatable member coupled to the distal end of the endoscopic hood, wherein the inflatable member is moveable between a deflated retracted configuration and an inflated extended configuration; and a conduit disposed alongside the elongate body of the endoscope, the conduit comprising a proximal end, and a distal end, wherein the distal end of the conduit is fluidly connected to a lumen of the inflatable member. The inflatable member may comprise a non-compliant or semi-compliant (e.g., semi-rigid) material, including, by way of non-limiting example, PEBAX, PET, PEN, PBT, PEEK, Hytrel, polyurethane and nylon. The non-compliant or semi-compliant material may be transparent. The inflatable member may include a balloon. The distal end of the inflatable member may be substantially longitudinally coextensive with the distal end of the endoscope when the inflatable member is in the deflated retracted configuration. The inflatable member may comprise one or more folds when in the deflated retracted configuration. At least a portion of the inflatable member may extend distally beyond the distal end of the endoscope when the inflatable member is in the inflated extended configuration. The portion of the inflatable member that extends distally beyond the distal end of the endoscope may form a hollow cylinder. An inner or outer surface of the hollow cylinder may include a series of parallel and evenly spaced line segments. The proximal end of the conduit may be fluidly connected to a fluid source, including, by way of non-limiting example, a gas or a liquid. The conduit may include a hydraulic tube. Flowing a fluid into the lumen of the inflatable member may move the inflatable member into the extended configuration, and flowing a fluid out of the lumen of the inflatable member may move the inflatable member into the retracted configuration.

In another embodiment, the present disclosure provides an endoscopic system comprising an endoscope comprising an elongate body having a proximal end, and a distal end, wherein at least a portion of the distal end includes a threaded outer surface; an endoscopic hood coupled to the distal end of the endoscope, wherein endoscopic hood is moveable between a retracted configuration and an extended configuration, the endoscopic hood comprising a proximal end, a distal end, a threaded inner surface, and a lumen extending between the proximal and distal ends, wherein the lumen is configured to receive the distal end of the endoscope such that the threaded outer surface of the endoscope rotatably engages the threaded inner surface of the endoscopic hood, an actuator disposed alongside the elongate body of the endoscope, the actuator comprising a proximal end, and a distal end, wherein the distal end of the actuator is coupled to the proximal end of the endoscopic hood and where the proximal end of the actuator is rotatable by user.

The endoscopic hood may comprise a transparent material, including, by way of non-limiting example, polymers including polyethylene, polyethylene terephthalate (PET), high-density polyethylene (HDPE), low density polyethylene (LDPE), polyvinyl chloride (PVC), polypropylene, polystyrene, polyester, polycarbonate, polyethersulfone, polyacrylate (PC) and polyetherketone (PEEK).

Rotating the proximal end of the actuator in a first direction may move the endoscopic hood distally along the threaded outer surface of the endoscope. Rotating the proximal end of the actuator in a second direction may move the endoscopic hood proximally along the threaded outer surface of the endoscope. A portion of the endoscopic hood may extend distally beyond the distal end of the endoscope when in the extended configuration. The portion of the endoscopic hood that extends distally beyond the distal end of the endoscope may be in the form of a hollow cylinder. An inner or outer surface of the hollow cylinder may include a series of parallel and evenly spaced line segments. The portion of the endoscopic hood that extends distally beyond the distal end of the endoscope may be unthreaded. The distal end of the endoscopic hood may be substantially longitudinally coextensive with the distal end of the endoscope when in the retracted configuration. The actuator may include a sheath disposed about a length of the endoscope body. The actuator may include a plurality of wires extending longitudinally along the elongate body. The actuator may further comprise a plurality of rings extending around the elongate body, wherein the rings are longitudinally spaced along a length of the elongate body, and wherein the plurality of wires are attached to the rings.

In another embodiment, the present disclosure provides a method of examining a body passageway, comprising inserting, into a body lumen of a patient, an endoscopic system comprising an endoscope comprising an elongate body having a proximal end and a distal end; an endoscopic hood coupled to the distal end of the endoscope, the endoscopic hood comprising a proximal end, a distal end, and a lumen extending therebetween, wherein the lumen is configured to receive the distal end of the endoscope; a flexible inflatable member coupled to the distal end of the endoscopic hood, wherein the inflatable member is moveable between a deflated retracted configuration and an inflated extended configuration; and a conduit disposed alongside the elongate body of the endoscope, the conduit comprising a proximal end, and a distal end, wherein the distal end of the conduit is fluidly connected to a lumen of the inflatable member, positioning a distal end of the endoscopic system adjacent a target tissue; moving the inflatable member from the retracted configuration to the extended configuration such that the inflatable member exerts a force against a fold in the target tissue, thereby exposing an obscured portion of the target tissue; and visualizing the obscured portion of the target tissue through the inflatable member. The method may further comprise moving the inflatable member from the extended configuration to the retracted configuration and repositioning the endoscope adjacent a different target tissue.

In another embodiment, the present disclosure provides a method of examining a body passageway, comprising inserting, into a body lumen of a patient, an endoscopic system comprising an endoscope comprising an elongate body having a proximal end, and a distal end, wherein at least a portion of the distal end includes a threaded outer surface; an endoscopic hood coupled to the distal end of the endoscope, wherein endoscopic hood is moveable between a retracted configuration and an extended configuration, the endoscopic hood comprising a proximal end, a distal end, a threaded inner surface, and a lumen extending between the proximal and distal ends, wherein the lumen is configured to receive the distal end of the endoscope such that the threaded outer surface of the endoscope rotatably engages the threaded inner surface of the endoscopic hood, an actuator disposed alongside the elongate body of the endoscope, the actuator comprising a proximal end, and a distal end, wherein the distal end of the actuator is coupled to the proximal end of the endoscopic hood and where the proximal end of the actuator is rotatable by user; positioning the endoscopic system adjacent a target tissue; moving the endoscopic hood from the retracted configuration to the extended configuration such that the distal end of the endoscopic hood exerts a force against a fold in the target tissue, thereby exposing an obscured portion of the target tissue; and visualizing the obscured portion of the target tissue through the endoscopic hood. The method may further comprise moving the endoscopic hood from the extended configuration to a retracted configuration and repositioning the endoscope adjacent a different target tissue.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 depicts an endoscopic hood that includes an inflatable member in a retracted configuration, according to one embodiment of the present disclosure.

FIG. 2 depicts the endoscopic hood of FIG. 1 on the distal end of an endoscope, according to another embodiment of the present disclosure.

FIG. 3 depicts the endoscopic hood of FIG. 1 on the distal end of an endoscope with the inflatable member in an extended configuration, according to another embodiment of the present disclosure.

FIG. 4 depicts an endoscopic hood that includes a threaded inner surface, according to another embodiment of the present disclosure.

FIG. 5 depicts the endoscopic hood of FIG. 4 in a retracted configuration on the distal end of an endoscope, according to another embodiment of the present disclosure.

FIG. 6 depicts the endoscopic hood of FIG. 4 in an extended configuration on the distal end of an endoscope, according to another embodiment of the present disclosure.

FIG. 7 depicts an alternative embodiment of an endoscopic hood that includes a threaded inner surface, according to another embodiment of the present disclosure.

FIGS. 8A-8B depict an endoscopic hood on the distal end of an endoscope positioned within the GI tract, with the inflatable member in a retracted configuration adjacent to a lesion hidden within a fold of the lumen wall (FIG. 8A), and the inflatable member in an extended configuration exposing the hidden lesion (FIG. 8B), according to one embodiment of the present disclosure.

FIGS. 9A-9B depict an endoscopic hood on the distal end of an endoscope positioned within the GI tract, with the endoscopic hood in a retracted configuration adjacent to a lesion hidden within a fold of the lumen wall (FIG. 9A), and the endoscopic hood in an extended configuration exposing the hidden lesion (FIG. 9B), according to another embodiment of the present disclosure.

It is noted that the drawings are intended to depict only typical or exemplary embodiments of the disclosure. It is further noted that the drawings may not be necessarily to scale. Accordingly, the drawings should not be considered as limiting the scope of the disclosure. The disclosure will now be described in greater detail with reference to the accompanying drawings.

### DETAILED DESCRIPTION OF THE PREFERRED EMBODIMENTS

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to an endoscope hood attached to the distal end of an endoscope, the endoscope hood disclosed herein may be attached to a variety of medical devices that are inserted into a variety of lumens of a patient, including for example, guide lumens, ports, optical wands and the like. As used herein, the term “distal” refers to the end farthest away from a medical professional when introducing a device into a patient, while the term “proximal” refers to the end closest to the medical professional when introducing a device into a patient.

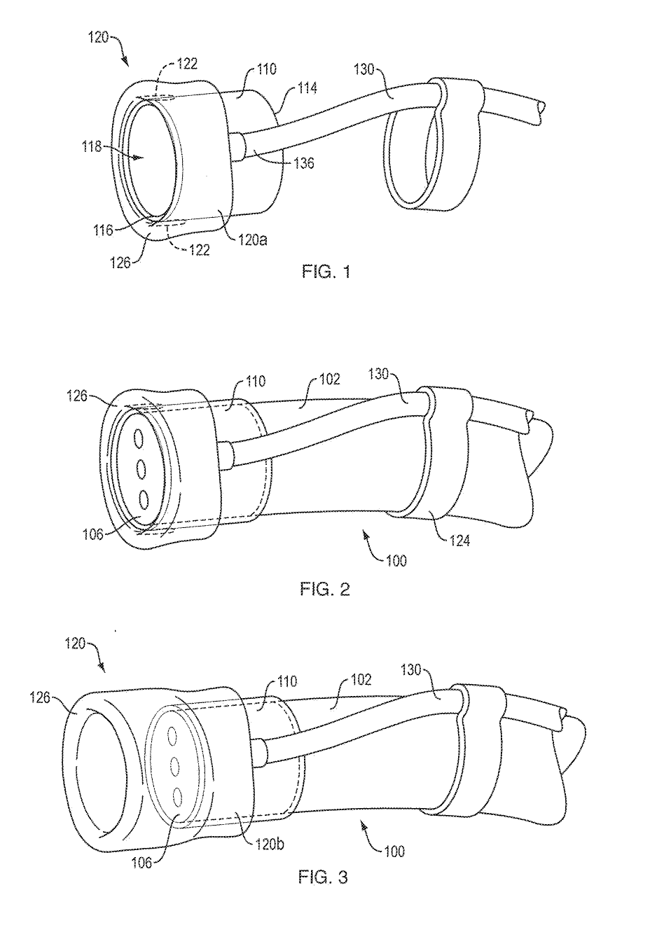

In one embodiment, the present disclosure provides a transparent retractable extension (TRE) endoscopic system for visualizing small and/or hidden lesions or tumors within the lumen wall of the GI tract. As illustrated in FIG. 1, in one embodiment the endoscopic system of the present disclosure may include an endoscopic hood 110 comprising a proximal end 114, a distal end 116 and a lumen 118 extending therebetween. A flexible inflatable member 120 (e.g. a balloon) is coupled to the distal end 116 of the endoscopic hood 110. A conduit 130 comprising a distal end 136 is disposed along an outer surface of the endoscopic hood 110, such that the distal end 136 of the conduit 130 is fluidly connected to a lumen of the flexible inflatable member 120. The proximal end (not shown) of the conduit 130 is fluidly connected to a fluid source (not shown) to deliver an inflation fluid into the lumen of the flexible inflatable member. The inflation fluid may include a variety of physiologically inert liquids (e.g., buffered solutions such as sterile saline) or gases (e.g., oxygen, nitrogen, hydrogen, carbon dioxide, helium etc.) as are known in the art. The flexible inflatable member 120 may be moveable from a deflated retracted configuration 120a comprising one or more folds 122, to an inflated extended configuration 120b (FIG. 3) by flowing the inflation fluid from the fluid source into the lumen of the flexible inflatable member 120. Similarly, the flexible inflatable member 120 may be moveable from an inflated extend configuration 120b to a deflated retracted configuration 120a by flowing the inflation fluid from the lumen of the flexible inflatable member 120 back to the fluid source.

The flexible inflatable member 120 may include a combination of elastomeric and semi to non-compliant materials that lack elastic properties and can only assume one shape when in the expanded configuration. In one embodiment, the non-compliant or semi-compliant material of the flexible inflatable member 120 is transparent. For example, flexible inflatable member 120 may include one or more thermoplastics and/or thermosets. Examples of thermoplastics include polyolefins; polyamides (e.g., nylon, such as nylon 12, nylon 11, nylon 6/12, nylon 6, nylon 66); polyesters (e.g., polyethylene terephthalate (PET), polybutylene terephthalate (PBT), polyethylene naphthalate (PEN), polytrimethylene terephthalate (PTT)); polyethers; polyurethanes; polyvinyls; polyacrylics; fluoropolymers; copolymers and block copolymers thereof, such as block copolymers of polyether and polyamide (e.g., PEBAX®); and mixtures thereof. Examples of thermosets include elastomers (e.g., EPDM), epichlorohydrin, polyureas, nitrile butadiene elastomers and silicones. Other examples of thermosets include epoxies and isocyanates. Biocompatible thermosets may also be used. Biocompatible thermosets include, for example, biodegradable polycaprolactone, poly(dimethylsiloxane) containing polyurethanes and ureas and polysiloxanes. Ultraviolet curable polymers, such as polyimides and acrylic or methacrylic polymers and copolymers may also be used.

Referring to FIG. 2, the lumen 118 of the endoscopic hood 110 may be configured to receive (e.g., via frictional or interference fit) the distal end 106 of an endoscope 100. The conduit 130 and endoscopic hood 110 may be further secured longitudinally along the elongate body 102 of the endoscope 100 by one or more clips 124. While the clip 124 of FIG. 2 engages a full circumference of the outer surface of the elongate body 102, a variety of clip configuration are contemplated by the present disclosure, including, for example, clips that only engage a portion of the outer surface of the elongate body. To facilitate navigation within the tortuous anatomy of the GI tract, the flexible inflatable member 120 may be substantially longitudinally coextensive (e.g., flush) with the distal end 106 of the endoscope 100 when in the deflated retracted configuration 120a. As illustrated in FIG. 3, the flexible inflatable member 120 may move from a deflated retracted configuration 120a (FIG. 2) to an inflated extended configuration 120b by flowing an inflation fluid from the fluid source (not shown) through conduit 130 into the lumen of the flexible inflatable member 120 such that at least a portion of the flexible inflatable member 120 forms a hollow cylinder that extends distally beyond the distal end 106 of the endoscope 100. In one embodiment, the hollow cylinder may include a series of parallel and evenly spaced line segments (not shown) to allow the physician to monitor how far the distal end 126 of the inflatable member 120 extends beyond the distal end 106 of the endoscope 100. As will be understood by one of skill in the art, the line segments (e.g., marked lines, hatch marks, hash marks, tick marks, etc.) may be formed, etched, scribed and/or drawn on an inner or outer surface of the hollow cylinder.

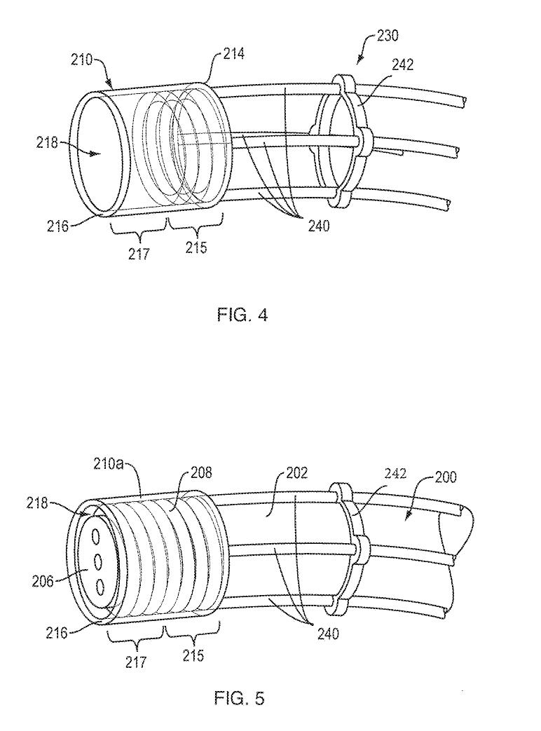

As illustrated in FIG. 4, in another embodiment the endoscopic system of the present disclosure may include an endoscopic hood 210 comprising a proximal end 214, a distal end 216 and a lumen 218 extending therebetween. The endoscopic hood 210 may be formed from a translucent or transparent material, such as a clear polymer-based material (e.g., clear plastics, etc.) as are known in the art. In one embodiment, a proximal portion of the endoscopic hood 210 defining the lumen 218 may include a threaded inner surface 215 while a distal portion of the endoscopic hood 210 defining the lumen 218 may be unthreaded 217 (e.g., smooth). An actuator 230 may extend proximally from the proximal end 214 of the endoscopic hood 210. In one embodiment, the actuator 230 may include a plurality of wires/cables 240 connected to each other by one or more rings 242.

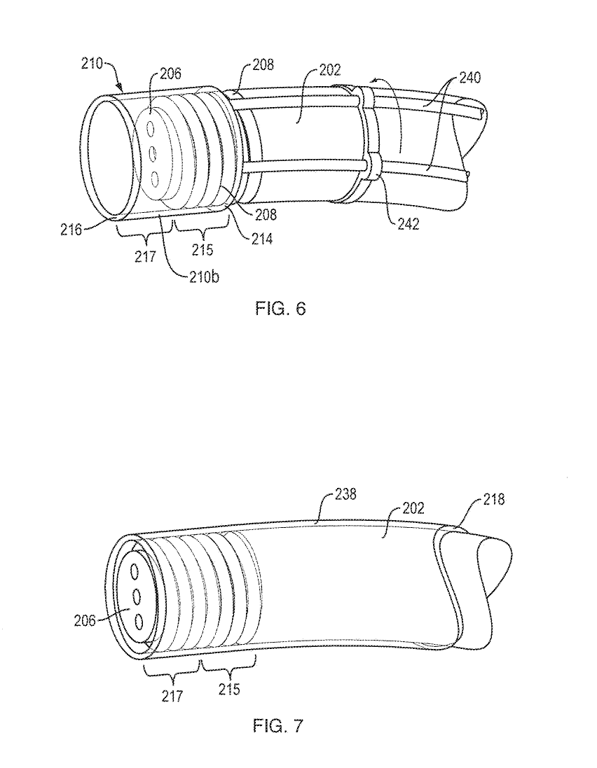

Referring to FIG. 5, the lumen 218 of the endoscopic hood 110 may be configured to receive the distal end 206 of an endoscope 200. In one embodiment, a portion of the distal end 206 of the endoscope 200 may include a threaded outer surface 208 configured to rotatably engage the corresponding threaded inner surface 215 of the endoscopic hood 210. The plurality of wires/cables 240 of the actuator 230 may extend longitudinally along the elongate body 202 of the endoscope 200. One or more rings 242 may be longitudinally spaced along the length of the elongate body 202 of the endoscope 200 to connect the plurality of wire/cables 240. The wire/cables 240 and rings 242 may be formed from may sufficiently flexible and torsionally compliant materials (e.g., shape-memory polymers and/or shape-memory metals as are known in the art) to bend and/or flex as the endoscope is advanced and/or retracted through a body lumen of the patient, while still being able to translate rotational force at the proximal end (not shown) of the actuator 230 to the endoscopic hood 210. To facilitate navigation within the tortious anatomy of the GI tract, the endoscopic hood 210 may be substantially longitudinally coextensive (e.g., flush) with the distal end 206 of the endoscope 200 when in the retracted configuration 210a. As illustrated in FIG. 6, the endoscopic hood 210 may move from a retracted configuration 210a (FIG. 5) to an extended configuration 210b by rotating the proximal end (not shown) of actuator 230 in a first (e.g., clockwise) direction along the threaded outer surface 208 of the endoscope 200 such that at least a portion of the endoscopic hood 210 forms a hollow cylinder that extends distally beyond the distal end 206 of the endoscope 200. In one embodiment, as discussed above, an inner and/or outer surface of the hollow cylinder may include a series of parallel and evenly spaced line segments (not shown) to allow the physician to monitor how far the distal end 216 of the endoscopic hood 210 extends beyond the distal end 206 of the endoscope 200. Still referring to FIG. 6, in one embodiment, the portion of the endoscopic hood 210 extending distally beyond the distal end 206 of the endoscope 200 may include a smooth (e.g., unthreaded) inner surface 217, thereby minimizing surface area for intestinal debris to collect within the lumen 218 of the endoscopic hood 210. The endoscopic hood 210 may be moveable from the extended configuration (FIG. 6) to the retracted configuration (FIG. 5) by rotating the proximal end (not shown) of actuator 230 in a second (e.g., counter-clockwise) direction along the threaded outer surface 208 of the endoscope 200.

Referring to FIG. 7, in another embodiment the actuator may include an elongate sheath 238 disposed about and extending longitudinally along the length of the elongate body 202 of the endoscope 200. As with actuator 230, the endoscopic hood may be moved from the retracted configuration to the extended configuration by rotating the proximal end (not shown) of the elongate sheath 238 in a first (e.g., clockwise) direction along the threaded outer surface 208 of the endoscope 200, and from the extended configuration to the retracted configuration by rotating the proximal end (not shown) of the elongate sheath 238 in a second (e.g., counter-clockwise) direction along the threaded outer surface 208 of the endoscope 200.

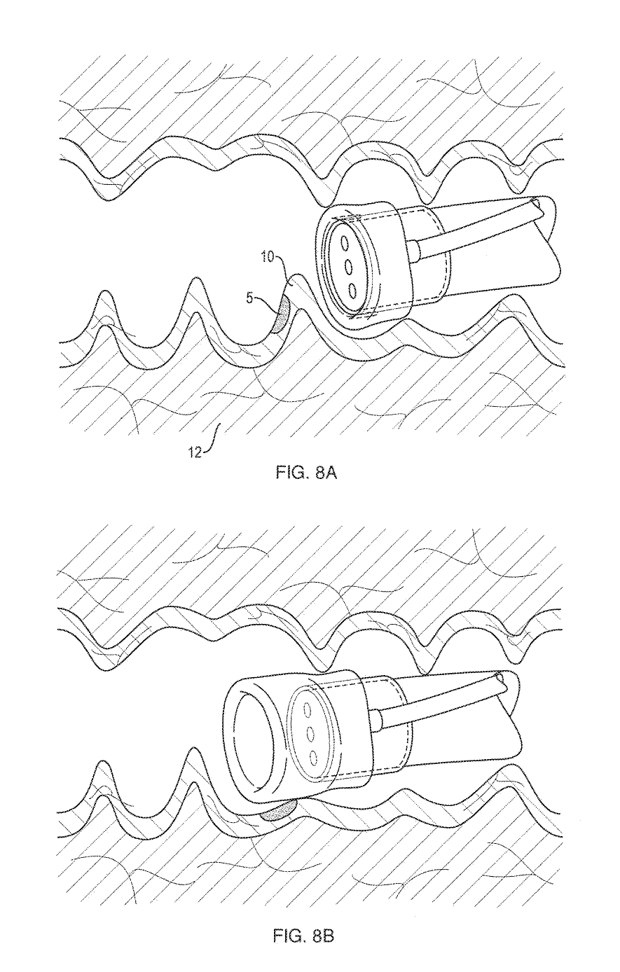

As illustrated by FIGS. 8A-8B, in use and by way of example, the endoscopic system of FIG. 2 may be inserted into a body lumen (e.g., GI tract) of a patient such that the distal end 106 of the endoscope 100 is positioned adjacent a target tissue that includes one or more folds 10 that the physician believes to be hiding or otherwise obstructing visualization of a lesion 5. The flexible inflatable member 120 may then be moved from the deflated retracted configuration (FIG. 8A) to an inflated extended configuration (FIG. 8B) as explained above such that the distal end 126 of the flexible inflatable member 120 extends distally beyond the distal end 106 of the endoscope 100. When in the inflated extended configuration the distal end 126 of the flexible inflatable member 120 exerts a longitudinal force against one or more folds of the lumen wall, thereby stretching and exposing the target tissue hidden or obscured within the fold. The physician may then visualize the target tissue, which may include a lesion, through the transparent material of the flexible inflatable member 120. If the exposed tissue does in fact include a lesion, the physician may then resect the tissue using methods known in the art, or alternatively, make note of the lesion for resection by a subsequent interventional procedure. The physician may then return the flexible inflatable member 120 to the deflated retracted configuration (FIG. 8A) as discussed above, and reposition the distal end 106 of the endoscope 100 adjacent to the fold of another target tissue and repeat the steps outlined above. In addition to exposing tissues within the folds of the lumen wall, moving the flexible inflatable member 120 from the inflated extended configuration to the deflated retracted configuration allows intestinal debris that may have become lodged within the hollow cylindrical opening of the flexible inflatable member to be cleared from the endoscope.

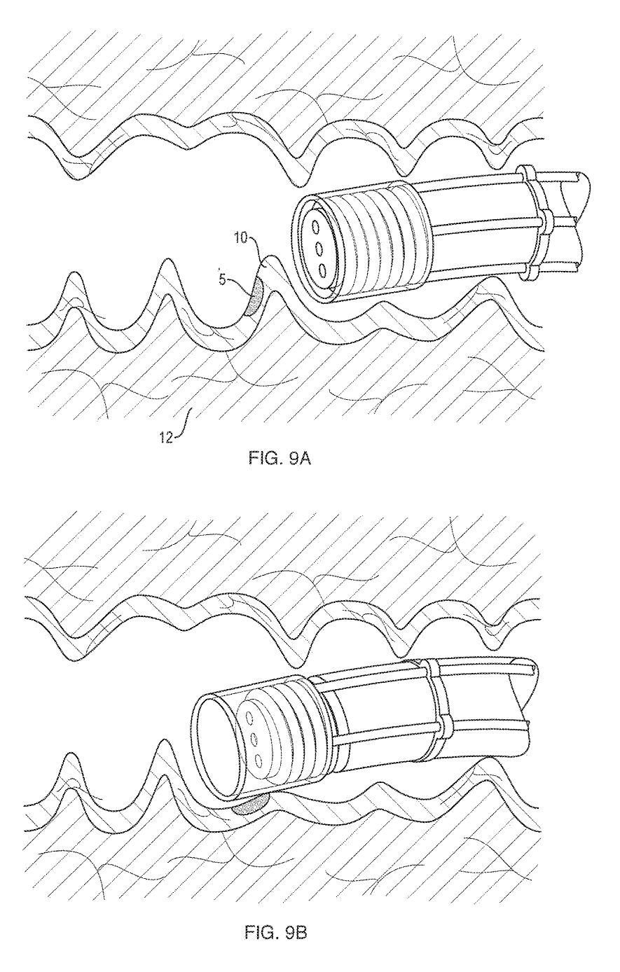

As illustrated by FIGS. 9A-9B, in use and by way of example, the endoscopic system of FIG. 5 may be inserted into a body lumen (e.g., GI tract) of a patient such that the distal end 206 of the endoscope 200 is positioned adjacent a target tissue that includes one or more folds 10 that the physician believes to be hiding or otherwise obstructing visualization of a lesion 5. The endoscopic hood 210 may then be moved from the retracted configuration (FIG. 9A) to an extended configuration (FIG. 9B) as explained above such that the distal end 216 of the endoscopic hood 210 extends distally beyond the distal end 206 of the endoscope 200. When in the extended configuration the distal end 216 of the endoscopic hood 210 exerts a longitudinal force against one or more folds of the lumen wall, thereby stretching and exposing the target tissue hidden or obscured within the fold. The physician may then visualize the target tissue, which may include a lesion, through the transparent material of the endoscopic hood. If the exposed tissue does in fact include a lesion, the physician may then resect the tissue using methods known in the art, or alternatively, make note of the lesion for resection by a subsequent interventional procedure. The physician may then return the endoscopic hood 210 to the retracted configuration (FIG. 9A) as discussed above, and reposition the distal end 206 of the endoscope 200 adjacent to the fold of another target tissue and repeat the steps outlined above. In addition to exposing tissues within the folds of the lumen wall, moving the endoscopic hood 210 from the extended configuration to the retracted configuration allows intestinal debris that may have become lodged within the hollow cylindrical opening of the flexible inflatable member to be cleared from the endoscope. Accumulation of intestinal debris within the endoscopic hood may be further minimized by the smooth (e.g., unthreaded) inner surface 217 of the endoscopic hood which extends distally beyond the distal end 206 of the endoscope 200.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. An endoscopic system comprising:
an endoscope comprising an elongate body having:
a proximal end, and
a distal end;
an endoscopic hood coupled to the distal end of the endoscope, the endoscopic hood comprising:
a proximal end,
a distal end, and
a lumen extending therebetween, wherein the lumen is configured to receive the distal end of the endoscope;
an inflatable member coupled to the distal end of the endoscopic hood, wherein the inflatable member is moveable between a deflated retracted configuration and an inflated extended configuration; and
a conduit disposed alongside the elongate body of the endoscope, the conduit comprising:
a proximal end, and
a distal end, wherein the distal end of the conduit is fluidly connected to a lumen of the inflatable member.

2. The endoscopic system of claim 1, wherein the inflatable member comprises a non-compliant or semi-compliant material selected from the group consisting of PEBAX, PET, PEN, PBT, PEEK, Hytrel, polyurethane and nylon.

3. The endoscopic system of claim 1, wherein the inflatable member comprises a transparent material.

4. The endoscopic system of claim 1, wherein a distal end of the inflatable member is substantially longitudinally coextensive with the distal end of the endoscope when the inflatable member is in the deflated retracted configuration.

5. The endoscopic system of claim 1, wherein at least a portion of the inflatable member extends distally beyond the distal end of the endoscope when the inflatable member is in the inflated extended configuration.

6. The endoscopic system of claim 1, wherein the proximal end of the conduit is fluidly connected to a fluid source.

7. The endoscopic system of claim 1, wherein flowing a fluid into the lumen of the inflatable member moves the inflatable member into the inflated extended configuration.

8. The endoscopic system of claim 1, wherein flowing a fluid out of the lumen of the inflatable member moves the inflatable member into the deflated retracted configuration.

9. An endoscopic system comprising:
an endoscope comprising an elongate body having:
a proximal end, and
a distal end, wherein at least a portion of the distal end includes a threaded outer surface;
an endoscopic hood coupled to the distal end of the endoscope, wherein endoscopic hood is moveable between a retracted configuration and an extended configuration, the endoscopic hood comprising:
a proximal end,
a distal end,
a threaded inner surface, and
a lumen extending between the proximal and distal ends, wherein the lumen is configured to receive the distal end of the endoscope such that the threaded outer surface of the endoscope rotatably engages the threaded inner surface of the endoscopic hood; and
an actuator disposed alongside the elongate body of the endoscope, the actuator comprising:
a proximal end, and
a distal end, wherein the distal end of the actuator is coupled to the proximal end of the endoscopic hood and where the proximal end of the actuator is rotatable by user.

10. The endoscopic system of claim 9, wherein the endoscopic hood comprises a transparent material selected from the group consisting of polyethylene, polyethylene terephthalate (PET), high-density polyethylene (HDPE), low density polyethylene (LDPE), polyvinyl chloride (PVC), polypropylene, polystyrene, polyester, polycarbonate, polyethersulfone, polyacrylate (PC) and polyetherketone (PEEK).

11. The endoscopic system of claim 9, wherein rotating the proximal end of the actuator in a first direction moves the endoscopic hood distally along the threaded outer surface of the endoscope.

12. The endoscopic system of claim 9, wherein rotating the proximal end of the actuator in a second direction moves the endoscopic hood proximally along the threaded outer surface of the endoscope.

13. The endoscopic system of claim 9, wherein a portion of the endoscopic hood extends distally beyond the distal end of the endoscope when in the extended configuration.

14. The endoscopic system of claim 13, wherein the portion of the endoscopic hood that extends distally beyond the distal end of the endoscope is unthreaded.

15. The endoscopic system of claim 9, wherein the distal end of the endoscopic hood is substantially longitudinally coextensive with the distal end of the endoscope when in the retracted configuration.

16. The endoscopic system of claim 9, wherein the actuator includes a sheath disposed about a length of the endoscope elongate body.

17. The endoscopic system of claim 9, wherein the actuator includes a plurality of wires extending longitudinally along the elongate body.

18. The endoscopic system of claim 17, wherein the actuator further comprises a plurality of rings extending around the elongate body, wherein the rings are longitudinally spaced along a length of the elongate body, and wherein the plurality of wires are attached to the rings.

19. A method of examining a body passageway, comprising:
a) inserting, into a body lumen of a patient, an endoscopic system;,
b) positioning a distal end of the endoscopic system adjacent a target tissue;
c) moving an inflatable member from the deflated retracted configuration to the inflated extended configuration such that the inflatable member exerts a force against a fold in the target tissue, thereby exposing an obscured portion of the target tissue; and
d) visualizing the obscured portion of the target tissue through the inflatable member.

20. The method of claim 19, further comprising:
e) moving the inflatable member from the extended configuration to the retracted configuration and
f) repositioning the endoscope adjacent a different target tissue.
